(19) United States
(12) Patent Application Publication
(10) Pub. No.: US 2005/0075563 A1
(43) Pub. Date: Apr. 7, 2005

(54) CT imaging system for robotic intervention

(76) Inventors: Predrag Sukovic, Ann Arbor, MI (US); Neal Clinthomc, Ann Arbor, MI (US)

Correspondence Address: CARLSON, GASKEY & OLDS, P.C., 400 WEST MAPLE ROAD, SUITE 350, BIRMINGHAM, MI, 48009, US

(21) Appl. No.: 10/958,179

(22) Filed: Oct. 4, 2004

Related U.S. Application Data
(60) Provisional application No. 60/508,517, filed on Oct. 3, 2003.

(51) Int. Cl. A61B 5/05

(52) U.S. Cl. 600/427

(57) ABSTRACT

The present invention is an image-guided robotic surgical system including a CT scanning system. The CT scanning system first performs a low-dose scan of a general area of interest of the patient's body and an image is generated on a display. Using the image a region-of-interest, within the patient's body is defined. Limited field-of-view scans are used to update the region-of-interest image while data gathered during the initial scan is used for area outside the region-of interest.

### BACKGROUND OF THE INVENTION

For certain surgical treatments, the precise location of an instrument, such as a probe or needle, within a patient's body is critical. For example, particular medicines can be delivered via a needle to a precise location within the body. One such application is the delivery of an anti-cancer drug to the exact location of the tumor.

Doctors have used fluoroscopy to track the position of the needle into the body as it is inserted to the desired location. However, fluoroscopy only provides the doctors with a two-dimensional view of the needle's position in the body. As a result, it has been proposed to use a computed tomography scanner in order to provide a three-dimensional view of the position of the needle as the doctor inserts it into the body. The CT scanner operates continuously in order to provide an up-to-date three-dimensional view of the needle's position.

However, both the fluoroscopy and the CT scanner expose the doctor and the patient to radiation. Therefore, it has also been proposed to use robots, remotely controlled by the surgeon watching the CT image, to insert the needle into the patient's body. In these systems, the CT scanner includes an x-ray source and an x-ray detector on opposite sides of the patient's body near the needle. The x-ray from the x-ray source is collimated to emit a fan-beam x-ray producing a plurality of “slices” through the patient's body as the x-ray source and detector revolve around the patient's body. The doctor views the three-dimensional image while remotely controlling the needle's position in the patient's body. In this manner, the doctor can avoid the unnecessary doses of radiation.

This proposed CT system has some drawbacks. First, because the x-ray source is a fan-beam x-ray source, imaging only a narrow slice at a time, it is difficult to keep the tip of the needle in the field of view. This is particularly true when the needle is traveling generally parallel to the axis of rotation of the CT scanner. The CT scanner is fixed in the room, so the patient bed, the patient and the robot must be translated along the axis of rotation of the CT scanner to keep the needle tip in the field of view. Additionally, although the doctor can avoid excessive doses of radiation by using the robot, the continuous scanning by the CT scanner exposes the patient's body to more radiation than necessary.

### SUMMARY OF THE INVENTION

The present invention is an image-guided surgical system including a CT scanning system, for example, for use with robotic intervention.

The CT scanning system includes a source and detector mounted to a c-arm positioned on a carriage, such that the c-arm can be rotated about an axis centered within the c-arm. The carriage is also slidably mounted on rails such that the carriage and c-arm can translate along the axis. The system further includes a surgical robot for inserting a needle into a patient's body.

A controller controls the source, detector, surgical robot, and any hardware for moving the c-arm. The controller may be a CPU including a display and an input device. The CPU gathers the data and images from the detector and generates a three-dimensional image. The controller and the doctor controlling the system would be in a location that is shielded from radiation of the x-ray source.

The CT scanning system first scans a low-dose scan of the general area of interest of the patient's body and a three-dimensional model or image is generated by the CPU. Using the image and an input device, the doctor defines a region-of-interest, within the patient's body. Once the region-of-interest is defined, the source and detector are then activated to produce a plurality of images of the region-of-interest.

If it is assumed that the areas of the patient's body outside the region-of-interest are not going to change during the procedure, then it is sufficient to use the data gathered during the initial, low-dose scan for the areas of the patient's body surrounding the region-of-interest, to update the original model. Only limited field-of-view scans are needed to update the region-of-interest image. Additionally, the x-ray source is a cone-beam x-ray source to easier to keep the needle within the image during the region-of-interest scan. Thus, the patient's body receives a lower dose of radiation than would otherwise be applied.

### BRIEF DESCRIPTION OF THE DRAWINGS

The above, as well as other advantages of the present invention, will become readily apparent to those skilled in the art from the following detailed description of a preferred embodiment when considered in the light of the accompanying drawing in which:

FIG. 1 is a schematic of the surgical and CT scanning system of the present invention;

FIG. 2 illustrates an end view of the surgical and CT scanning system;

FIG. 3 illustrates an initial low dose scan of a general area; and

FIG. 4 illustrates a high dose region of interest scan.

### DETAILED DESCRIPTION OF THE PREFERRED EMBODIMENT

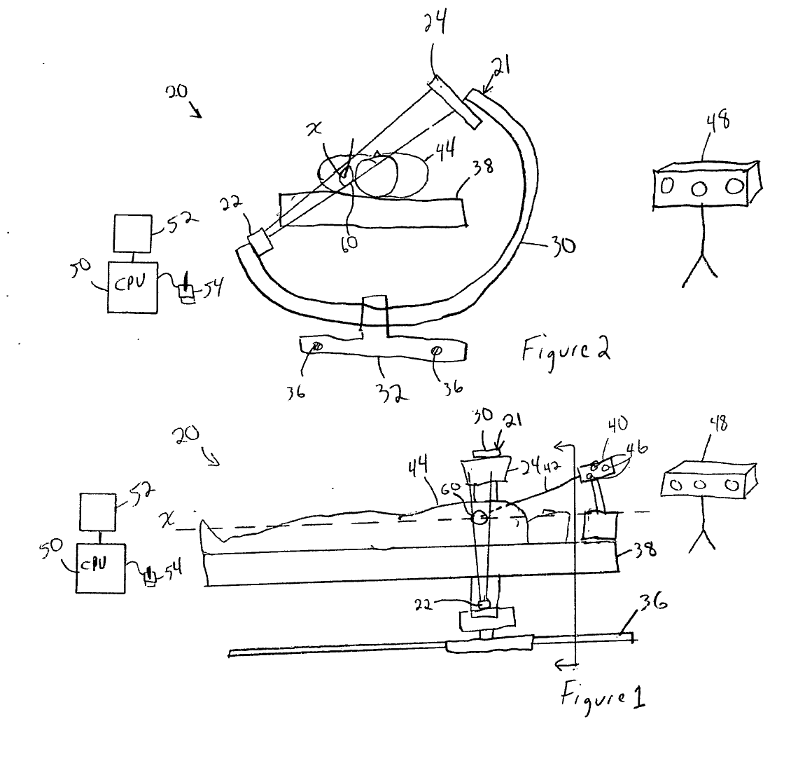

FIGS. 1 and 2 shows an image-guided robotic surgical system 20 including a CT scanning system 21. The CT scanning system 21 includes a source 22 and detector 24 mounted at outer ends of a c-arm 30. The source 22 is preferably a cone-beam x-ray source 22. The c-arm 30 is also preferably slidably mounted on a carriage 32, such that the c-arm 30 can be rotated about an axis x, substantially centered within the c-arm 30 and positioned substantially between the source 22 and detector 24. The carriage 32 is also slidably mounted on rails 36 such that the carriage 32 and c-arm 30 can translate along the x-axis. The carriage 32 and/or the rails 36 may be part of (or simply placed below) a radiolucent operating table 38.

The system 20 further includes a surgical robot 40 for inserting a needle 42 into a patient's body 44 and delivering a drug at a precisely determined location in the patient's body 44 through the needle 42. The robot 40, or a portion of the robot 40, may optionally include a plurality of locators 46. The position of each of the locators 46 is tracked by a tracking system 48 to determine the position and orientation of the robot 40 and needle 42. Suitable tracking systems 48 and locators 46 are known in the field of image-guided surgery. The locators 46 and tracking system 48 are not necessary in the present invention, because the three-dimensional position and orientation of the needle 42 relative to the patient's body 44 is tracked with the CT scanner, but may further aid in the placement of the needle 42 and/or the control of the robot 40.

The source 22, detector 24, surgical robot 40 (FIG. 1), tracking system 48 (if used), and any motors and controllers for rotating and translating the c-arm 30 are all controlled by a controller, which may be a CPU 50. The CPU 50 includes a display 52 and an input device 54, such as a mouse, keyboard, joystick, etc. The CPU 50 also gathers the data and images from the detector 24 and generates three-dimensional images based upon the data and images from the detector 24. The CPU 50, including display 52, input device 54, and the doctor controlling the system 20 via input device 54, would be in a location that is shielded from radiation of the x-ray source 22.

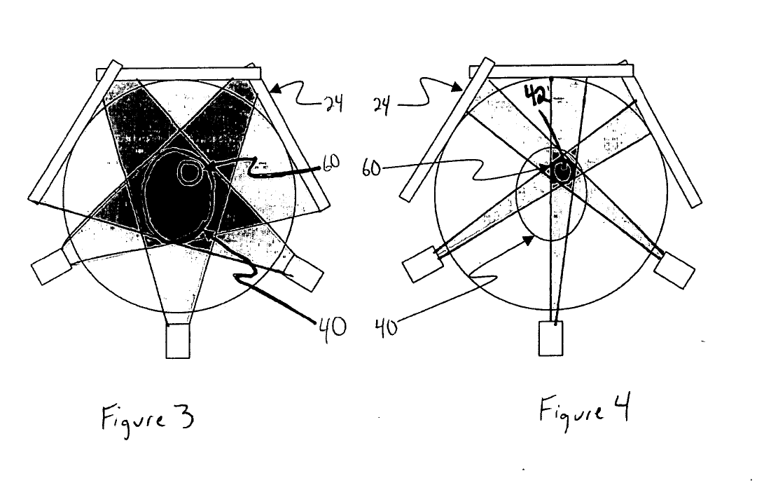

Referring also to FIG. 3, a low-dose scan of the general area of interest of the patient's body 44 (or the entire body 44) is first scanned by the CT scanning system 21 and a three-dimensional model or image is generated by the CPU 50 and displayed on the display 52. While viewing the image on the display 52 and by using the input device 54, the doctor defines a three-dimensional region-of-interest 60, such as a sphere, within the patient's body 44. For example, it is anticipated for the particular application of inserting a needle for drug delivery that the region-of-interest 60 would be on the order of a few inches in diameter.

Once the region-of-interest 60 is defined, the source 32 and detector 24 are then activated to produce a plurality of images of the region-of-interest 60 of the patient's body. The c-arm 30 is rotated about the x-axis by computer-controlled motors in the carriage 32 as the source 22 and detector 34 take images sufficient to update the three-dimensional image of the region-of-interest 60 of the patient's body. The doctor initiates the insertion of the needle 42 by the robot 40 into the patient's body 44 toward the region-of-interest 60. Within the region-of-interest 60, the doctor controls the insertion of the needle 42 while watching the display 52 continuously update the three-dimensional displayed position and orientation of the needle 42 within the body 44. During the procedure, the doctor can rotate, enlarge or otherwise manipulate the image on the display 52, so that the doctor can monitor, control and adjust the travel of the needle 42 into the body 44.

Referring to FIG. 4, since the CPU 50 has already stored data relating to the areas of the patient's body 44 surrounding the region-of-interest 60, the CPU 50 can update the original model of the region-of-interest 60 based upon the data from the initial, full scan and based upon the limited field-of-view scan of just the region-of-interest 60. As can be seen in FIG. 4, the cone beam x-ray from the x-ray source 22 is narrowed substantially, such that it does not pass through the entire portion of the patient's body, but is focused only on the region-of-interest 60. It is assumed that the areas of the patient's body outside the region-of-interest 60 are not going to change during the procedure, so it is sufficient to simply use the data gathered during the single, initial, low-dose full scan (FIG. 3). Thus, the patient's body 44 receives a lower dose of radiation than would otherwise be applied. Additionally, because the x-ray source 22 is a cone-beam x-ray source 22, it is easier to keep the needle 42 within the region-of-interest 60 during the procedure. When the needle 42 has reached the desired location, the doctor controls the robot 40 to deliver the drug and then retract out of the patient's body 44. Alternatively, information regarding the areas of the patient's body outside the region of interest 60 may be generic—i.e. predetermined and pre-stored and not specifically from the particular patient for which it is used.

The CT scanning system 21 of the present invention could also be used without the robot 40. The doctor could manually insert the needle 42 (or probe) into the patient's body 44 while monitoring the position and orientation of the needle 42 on the display 52 to ensure that the needle 42 is inserted into precisely the desired location within the patient's body 44.

Alternatively, or as an addition to the updates performed by the CT scanning system (i.e. between CT updates), the locators 46 and tracking system 48 may be used to track the position of the needle 42 relative to the 3-dimensional image of the patient's body 44 created from a CT scan. Similarly, sensors and motors in the robot 40 could provide the information regarding the position of the needle 42 relative to the 3-dimensional image as the needle 42 is inserted.

Although a preferred embodiment of this invention has been disclosed, a worker of ordinary skill in this art would recognize that certain modifications would come within the scope of this invention. For that reason, the following claims should be studied to determine the true scope and content of this invention.

1. A computer guided surgical system comprising:
a needle insertable into a region of interest in a patient;
a X-ray source and detector rotatable about an axis;
a controller connected to the X-ray source and detector in order to control scanning of the region of interest of the patient, the region of interest contained within a general area of the patient, the controller generating an updated 3D model of the region of interest and the needle based upon the scanning of the region of interest; and
a display connected to said controller to provide an updated image of the needle within the region of interest.

2. The computer guided surgical system of claim 1, wherein said display is continuously updated by repeatedly scanning the region of interest.

3. The computer guided surgical system of claim 1, wherein said controller controls movement of the needle within the region of interest.

4. The computer guided surgical system of claim 1, wherein the controller generates the updated 3D model of the region of interest based upon the scanning of the region of interest and based upon information regarding the general area of the patient surrounding the region of interest.

5. The computer guided surgical system of claim 4, wherein said X-ray source is a cone-beam X-ray source.

6. The computer guided surgical system of claim 1, wherein the information regarding the general area of the patient is a scan of the general area of the patient at a dosage lower than the scanning of the region of interest.

7. The computer guided surgical system of claim 1, wherein said controller includes a CPU and computer input device.

8. A method of computer guided surgery comprising:
a) storing first image information regarding a general area of a patient;
b) selecting an region of interest within the general area;
c) scanning the region of interest;
d) creating a three dimensional image of the region of interest based upon the first image information and based upon said step c); and
e) guiding a needle within the region of interest based upon the three dimensional image.

9. The method of claim 8, wherein said step a) further includes using a low dose scan of the general area of the patient to collect data for the first image information.

10. The method of claim 9, wherein said step c) further includes using a higher dosage scan than used for said step a).

11. The method of claim 8, wherein said step c) further includes using a conical shaped X-ray beam to scan the region of interest.

12. The method of claim 11, wherein a smaller diameter beam is used in said step c) than in said step a).

13. The method of claim 8, further including repeating steps c-d) during said step e), such that the guiding in said step e) is based upon an updated three dimensional image.

14. A computer guided surgical system comprising:
an X-ray source and detector mounted to a c-arm;
a carriage supporting said c-arm such that said c-arm can rotate about an axis at a center of said c-arm;
a pair of rails supporting said c-arm and carriage such that said c-arm and carriage can translate along said axis
a controller connected to the X-ray source, detector, and c-arm in order to scan an object, wherein said controller controls a needle within a region of interest in the object, the controller generating a three-dimensional image of the object based upon a general area scan of the object and based upon repeated scans of a region of interest in the object, the region of interest disposed within the general area; and
a display connected to said controller to provide an updated image of said object based upon the general area scan and the region of interest scans.

15. The computer guided surgical system of claim 14, wherein the region of interest is defined based upon the movement of the needle.
